(19) United States
(12) Patent Application Publication
(10) Pub. No.: US 2009/0238436 A1
(43) Pub. Date: Sep. 24, 2009

(54) COMPOSITION FOR IMAGING REAGENT

(75) Inventors: Jonathan A. Hickson, Lake Villa, IL (US); Scott L. Ackler, Gurnee, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, ABBOTT PARK, IL (US)

(21) Appl. No.: 12/407,283

(22) Filed: Mar. 19, 2009

Related U.S. Application Data
(60) Provisional application No. 61/037,766, filed on Mar. 19, 2008.

(51) Int. Cl. G06K 9/00; A61K 49/00; C09K 15/12

(52) U.S. Cl. 382/133; 424/9.6; 252/406

(57) ABSTRACT

The invention provides a composition comprising a buffer and an aminoluciferase bioluminescent imaging reagent. More specifically, the invention relates to a formulation for use with an aminoluciferase bioluminescent imaging reagent. The invention also provides a method for evaluating the efficacy of an anti-apoptotic agent over multiple time points comprising the in vivo use of a composition comprising a buffer and an aminoluciferase bioluminescent imaging reagent.

### CROSS-REFERENCE SECTION TO RELATED APPLICATION

This application claims the benefit of U.S. Provisional Patent Application No. 61/037,766, filed Mar. 19, 2008, which is hereby incorporated by reference in its entirety.

### FIELD OF THE INVENTION

The invention provides a composition comprising a buffer and an aminoluciferase bioluminescent imaging reagent. More specifically, the invention relates to a formulation for use with an aminoluciferase bioluminescent imaging reagent. The invention also provides a method for evaluating the efficacy of an anti-apoptotic agent over multiple time points comprising the in vivo use of a composition comprising a buffer and an aminoluciferase bioluminescent imaging reagent.

### BACKGROUND OF THE INVENTION

Non-invasively imaging apoptosis and other cellular processes of tumors in real time would be a useful tool for cancer researchers and clinicians. Methods of monitoring protease activity in vivo would provide a valuable tool for assessing preclinical drug efficacy, allowing for quicker development of cancer therapies. Rapid, sensitive cell-based assays providing sensitive luminescent methods to detect one or more protease exist. For instance, U.S. Pat. No. 7,148,030 describes a sensitive bioluminescent assay to detect proteases including caspases, trypsin and tryptase. Use of aminoluciferins for imaging is also known. Shah et al. (Molecular Therapy, Vol. 11, No. 6: 926-931) describes the single time-point use of a caspase-3-activatable aminoluciferin for imaging. One hurdle that has to be overcome with the in vivo use of aminoluciferase agents is the solubility of the agent. For use in imaging, a greater concentration of reagent may be needed than is necessary for in vitro use. When the reagent has a low solubility, it may only form a suspension or aggregation in solution at such higher concentrations. Aggregation may prevent reproducible delivery, cause irritation at the administration site or even cause a more systemic immunological response. Furthermore, in contrast to in vitro luminescent methods, it is important that an imaging agent be not only effective but also non-toxic. Such non-toxic solutions would allow researchers to evaluate an experimental compound's effectiveness in the same animal over multiple time points of treatment.

### SUMMARY OF THE INVENTION

The present invention provides a formulation for use with an aminoluciferase bioluminescent imaging reagent. In one embodiment, the formulation is a buffer for use with an aminoluciferase bioluminescent imaging reagent comprising between about 5 to 30% PEG 300 or 400, about 1 to 5% DMSO, about 1 to 15%, Tween 80, and about 50 to 93% aqueous solution. In another embodiment, the invention provides a buffer for use with an aminoluciferase bioluminescent imaging reagent in a rat, comprising between about 5 to 40% PEG 300 or 400, about 1 to 5% DMSO, about 1 to 15%, Tween 80, and about 40 to 93% aqueous solution. In another embodiment, the invention provides a buffer for use with an aminoluciferase bioluminescent imaging reagent in a mouse, comprising between about 5 to 40% PEG 300 or 400, about 1 to 5% DMSO, about 1 to 15%, Tween 80, and about 40 to 93% aqueous solution.

In another embodiment, the invention provides a composition comprising a buffer and an aminoluciferase bioluminescent imaging reagent. In another embodiment, the invention provides a composition comprising a buffer and an aminoluciferase bioluminescent imaging reagent, wherein the buffer comprises between about 5 to 30% PEG 300 or 400, about 1 to 5% DMSO, about 1 to 15%, Tween 80, and about 50 to 93% aqueous solution. In another embodiment, the invention provides a composition comprising a buffer and a negative control for an aminolucierin imaging agent, wherein the buffer comprises between about 30% PEG 300 or 400, about 5% DMSO, about 5%, Tween 80, and about 60% aqueous solution. In another embodiment, the invention provides a method for evaluating the efficacy of an anti-apoptotic agent over multiple time points comprising the in vivo use of a composition comprising a buffer and an aminoluciferase bioluminescent imaging reagent comprising
(a) administering to a subject luciferase-expressing cells;
(b) allowing the cells to achieve localization in the subject;
(c) administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(d) creating a first image of the photon emission of the subject with a photodetector device;
(e) at a later time-point, administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(f) creating a second image of the photon emission of the subject with a photodetector device; and
(g) comparing the photon emission of the first image and the second image.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All amino acid abbreviations used in this disclosure are those accepted by the United States Patent and Trademark Office as set forth in 37 C.F.R. sctn. 1.822(b).

As used herein, the term “between” when used to describe various ranges should be interpreted to include the end-points of the described ranges.

As used herein, the term “non-toxic” means a buffer that is reasonably safe when used in appropriate amounts and under appropriate conditions.

As used herein, the term “solution” means a solution that is clear upon visual inspection for at least an hour.

In another embodiment, the invention provides of a method for evaluating the efficacy of an anti-apoptotic agent comprising administering the buffered aminoluciferase bioluminescent imaging reagent to a mammal. In another embodiment, the mammal is a mouse. In another embodiment, the mammal is a rat.

### Embodiments of the Buffer

In one embodiment, the invention provides a buffer for use with an aminoluciferase bioluminescent imaging reagent. In a most preferred embodiment, the buffer is non-toxic. In a preferred embodiment, the composition is a solution. In one embodiment, the buffer comprises between about 5 to 30% PEG 300 or 400, about 1 to 5% DMSO, about 1 to 15%, Tween 80, and about 50 to 93% aqueous solution. In another embodiment, the buffer comprises between about 10 to 30% PEG 300 or 400, about 2 to 5% DMSO, about 1 to 10%, Tween 80, and about 55 to 87% aqueous solution. In another embodiment, the buffer comprises between about 25 to 30% PEG 300 or 400, about 3 to 5% DMSO, about 3 to 7%, Tween 80, and about 58 to 69% aqueous solution. In another embodiment, the buffer comprises between about 28 to 30% PEG 300 or 400, about 4 to 5% DMSO, about 4 to 6%, Tween 80, and about 59 to 64% aqueous solution. In another embodiment, the buffer comprises about 30% PEG 300 or 400, about 5% DMSO, about 5%, Tween 80, and about 60% aqueous solution. In another embodiment, the buffer comprises about 30% PEG 400, about 5% DMSO, about 5%, Tween 80, and about 60% a Dextrose 5% in Water (D5W).

In another embodiment, the buffer is for use in rat, and comprises between about 5 to 40% PEG 300 or 400, about 1 to 5% DMSO, about 1 to 15%, Tween 80, and about 40 to 93% aqueous solution. In another embodiment, the buffer comprises between about 10 to 40% PEG 300 or 400, about 2 to 5% DMSO, about 1 to 10%, Tween 80, and about 45 to 87% aqueous solution. In another embodiment, the buffer comprises between about 25 to 40% PEG 300 or 400, about 3 to 5% DMSO, about 3 to 7%, Tween 80, and about 48 to 69% aqueous solution. In another embodiment, the buffer comprises between about 28 to 40% PEG 300 or 400, about 4 to 5% DMSO, about 4 to 6%, Tween 80, and about 49 to 64% aqueous solution. In another embodiment, the buffer comprises about 40% PEG 300 or 400, about 5% DMSO, about 5%, Tween 80, and about 50% aqueous solution. In another embodiment, the buffer comprises about 40% PEG 400, about 5% DMSO, about 5%, Tween 80, and about 50% Dextrose 5% in Water (D5W).

In one embodiment, the aqueous solution is selected from the group consisting of water, saline, phosphate buffered saline, Hank's solution, Ringer's solution, dextrose/saline, glucose solutions and the like. In a preferred embodiment, the aqueous solution is Dextrose 5% in Water (D5W).

In one embodiment, the concentration of the aminoluciferase bioluminescent imaging reagent is about 18.75 to about 62.5 mg/ml.

### Embodiments of the Composition Comprising a Buffer and an Aminoluciferase Bioluminescent Imaging Reagent

In one embodiment, the invention provides a composition comprising a buffer and an aminoluciferase bioluminescent imaging reagent, wherein the buffer comprises between about 5 to 30% PEG 300 or 400, about 1 to 5% DMSO, about 1 to 15%, Tween 80, and about 50 to 93% aqueous solution. In another embodiment, the buffer is for use in rats, and comprises between about 10 to 40% PEG 300 or 400, about 2 to 5% DMSO, about 1 to 10%, Tween 80, and about 45 to 87% aqueous solution. In another embodiment, the buffer comprises about 30% PEG 300 or 400, about 5% DMSO, about 5%, Tween 80, and about 60% aqueous solution. In another embodiment, the buffer comprises about 30% PEG 400, about 5% DMSO, about 5%, Tween 80, and about 60% a Dextrose 5% in Water (D5W). In one embodiment, the aminoluciferase bioluminescent imaging reagent comprises an aminolucierin or a carboxy-terminal protected derivative thereof covalently linked via a peptide bond to a substrate for a protease. In another embodiment, the protease substrate is selected from the group consisting of a caspase, a trypsin and a tryptase. Preferred proteases that specifically cleave a substrate comprising aspartate include caspases. Even more preferred substrates include caspase 3 and 7. Preferred substrates comprise X₁-X₂-X₃-D, wherein X₁ is selected from the group consisting of Y, D, L, V, I, A, W and P; X₂ is selected from the group consisting of V and E, and X₃ is any amino acid. More preferably, X₁ is selected from the group consisting of A, V, H, I or T. Preferably, the substrate is selected from the group consisting of DEVD, WEHD, VDVAD, LEHD, VEID, VEVD, VEHD, IETD, AEVD, LEXD, VEXD, IEHD and PEHD. Even more preferably, the substrate is selected from the group consisting of DEVD, YVAD and LEHD. Even more preferably, the substrate comprises DEVD.

In another embodiment, the aminoluciferase bioluminescent imaging reagent comprises an aminolucierin or a carboxy-terminal protected derivative thereof covalently linked via a peptide bond to a kinase. In another embodiment, the aminoluciferase bioluminescent imaging reagent comprises an aminolucierin or a carboxy-terminal protected derivative thereof covalently linked via a peptide bond to a phosphatase. In another embodiment, the aminoluciferase bioluminescent imaging reagent comprises an aminolucierin or a carboxy-terminal protected derivative thereof covalently linked via a peptide bond to a transferase.

In one embodiment, composition remains in solution at a neutral pH. In another embodiment, the composition is suitable for intraperitoneal administration. In another embodiment, the composition is suitable for intravenous injection or infusion.

### Embodiments of the Composition Comprising a Buffer and a Negative Control for a Aminoluciferase Bioluminescent Imaging Reagent

In one embodiment, the invention provides a composition comprising a buffer and a negative control for an aminolucierin imaging agent, wherein the buffer comprises between about 30% PEG 300 or 400, about 5% DMSO, about 5%, Tween 80, and about 60% aqueous solution. In another embodiment, the buffer comprises about 30% PEG 400, about 5% DMSO, about 5%, Tween 80, and about 60% a Dextrose 5% in Water (D5W). In another embodiment, the buffer is for use in rats and comprises between about 10 to 40% PEG 400, about 2 to 5% DMSO, about 1 to 10%, Tween 80, and about 45 to 87% aqueous solution. In another embodiment, the negative control is a construct comprising an amino acid sequence attached to a luciferin substrate, wherein the amino acid sequence is not a substrate for either luciferase or a caspase.

In another embodiment, the construct is selected from the group consisting of

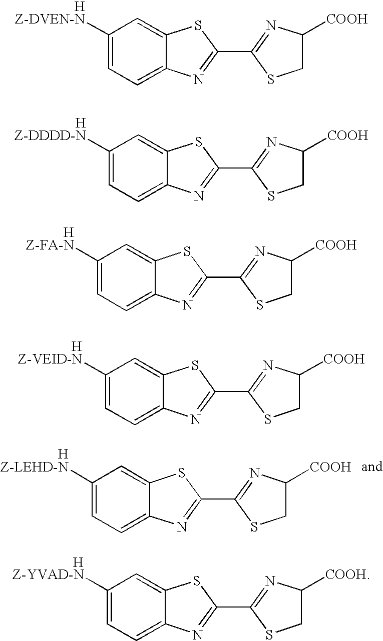

### Methods of Evaluating the Efficacy of an Anti-Apoptotic Agent

In one embodiment, the invention is directed to methods for evaluating the efficacy of an anti-apoptotic agent over multiple time points comprising the in vivo use of a composition comprising a buffer and an aminoluciferase bioluminescent imaging reagent comprising
(a) administering to a subject luciferase-expressing cells;
(b) allowing the cells to achieve localization in the subject;
(c) administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(d) creating a first image of the photon emission of the subject with a photodetector device;
(e) at a later time-point, administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(f) creating a second image of the photon emission of the subject with a photodetector device; and
(g) comparing the photon emission of the first image and the second image.

It is understood that additional administrations and imaging can be conducted under the above method. It is understood that the period of time elapsing between the creation of said first image and said second image can from as little as the time it takes to immediately administer the modulator and control to many days between images. In one embodiment, the localization of the luciferase-expressing cells takes from about 1 minute to about 1 hour. In another embodiment, the localization of the luciferase-expressing cells takes from about 5 minutes to about 30 minutes. In another embodiment, the buffer comprises about 30% PEG 400, about 5% DMSO, about 5%, Tween 80, and about 60% a Dextrose 5% in Water (D5W). In another embodiment, the aminoluciferase bioluminescent imaging reagent is DEVD-aminoluciferin.

### Experimentals

### Formulation for Suspending DEVD-Luciferin

The composition is as follows:

30% PEG 400

5% DMSO

5% Tween 80

60% Dextrose 5% in Water (D5W)

To formulate using this composition, the first three components can be combined ahead of time. Add the equivalent of 40% of the final solution volume dropwise to the powder while vortexing to wet the solution. Then add the remaining 60% of D5W dropwise while vortexing. Potentially need to quickly sonicate (10 seconds) to degas the solution. Using this formulation, we were able to achieve a solution of at least a 62.5 mg/ml.

### In Vivo Bioluminescent Imaging of VivoGlo™ Caspase-3/7 Substrate:

Mice will be injected with luciferase-expressing cells and allowed to localize. Once they reach a suitable size, the mouse will be treated with a known apoptosis-inducing agent. At timepoints post treatment, mice will be injected intraperitoneally with 62.5 mg/mL of VivoGlo™ Caspase-3/7 substrate in buffer comprising 30% PEG 400, 5% DMSO, 5%, Tween 80, and 60% a Dextrose 5% in Water (D5W). In luciferase-expressing cells undergoing apoptosis, caspase-3/7 will cleave the DEVD peptide from the aminolucifin, allowing it to be a substrate for luciferase photon production. The photon emission will be detected with a photodetector device from 5-30 minutes post injection and quantified.

We claim:

1. A buffer for use with an aminoluciferase bioluminescent imaging reagent comprising between about 5 to 30% PEG 300 or 400, about 1 to 5% DMSO, about 1 to 15%, Tween 80, and about 50 to 93% aqueous solution.

2. The buffer of claim 1, comprising between about 25 to 30% PEG 300 or 400, about 3 to 5% DMSO, about 3 to 7%, Tween 80, and about 58 to 69% aqueous solution.

3. The buffer of claim 1, comprising about 30% PEG 300 or 400, about 5% DMSO, about 5%, Tween 80, and about 60% aqueous solution.

4. The buffer of claim 1, wherein the aqueous solution is selected from the group consisting of water, saline, phosphate buffered saline, Hank's solution, Ringer's solution, dextrose/saline, glucose solutions and the like.

5. The buffer of claim 1, wherein the aqueous solution is Dextrose 5% in Water (D5W).

6. The buffer of claim 1, comprising about 30% PEG 400, about 5% DMSO, about 5%, Tween 80, and about 60% Dextrose 5% in Water (D5W).

7. The buffer of claim 1, wherein the buffer is non-toxic.

8. The buffer of claim 1, wherein the buffer is a solution.

9. A buffer for use with an aminoluciferase bioluminescent imaging reagent in a rat, comprising between about 5 to 40% PEG 300 or 400, about 1 to 5% DMSO, about 1 to 15%, Tween 80, and about 40 to 93% aqueous solution.

10. The buffer of claim 9, comprising about 40% PEG 400, about 5% DMSO, about 5%, Tween 80, and about 50% Dextrose 5% in Water (D5W).

11. A composition comprising a buffer and an aminoluciferase bioluminescent imaging reagent, wherein the buffer comprises between about 5 to 30% PEG 300 or 400, about 1 to 5% DMSO, about 1 to 15%, Tween 80, and about 50 to 93% aqueous solution.

12. The composition of claim 11, wherein the buffer comprises about 30% PEG 400, about 5% DMSO, about 5%, Tween 80, and about 60% a Dextrose 5% in Water (D5W).

13. The composition of claim 11, wherein the aminoluciferase bioluminescent imaging reagent comprises an aminoluciferin or a carboxy-terminal protected derivative thereof covalently linked via a peptide bond to a substrate for a protease.

14. The composition of claim 11, wherein the aminoluciferase bioluminescent imaging reagent comprises an aminoluciferin or a carboxy-terminal protected derivative thereof covalently linked via a peptide bond to a substrate for a caspase.

15. The composition of claim 14, wherein the substrate for the caspase is selected from the group consisting of DEVD, WEHD, VDVAD, LEHD, VEID, VEVD, VEHD, IETD, AEVD, LEXD, VEXD, IEHD and PEHD.

16. The composition of claim 14, wherein the substrate for the caspase is DEVD.

17. A composition comprising a buffer and a negative control for an aminolucierin imaging agent, wherein the buffer comprises between about 30% PEG 300 or 400, about 5% DMSO, about 5%, Tween 80, and about 60% aqueous solution.

18. The composition of claim 17, wherein the aminolucierin imaging agent is selected from the group of

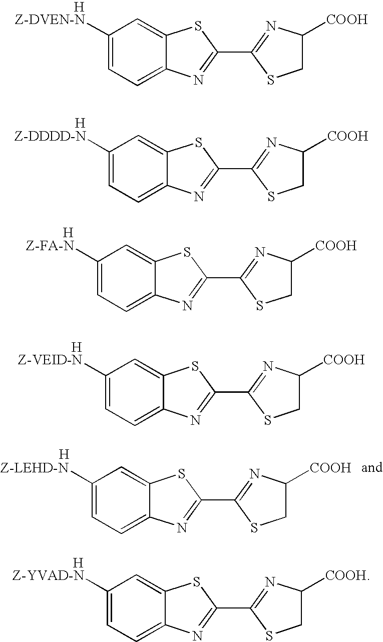

19. A method for evaluating the efficacy of an anti-apoptotic agent over multiple time points comprising the in vivo use of a composition comprising a buffer and an aminoluciferase bioluminescent imaging reagent comprising
(a) administering to a subject luciferase-expressing cells;
(h) allowing the cells to achieve localization in the subject;
(i) administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(j) creating a first image of the photon emission of the subject with a photodetector device;
(k) at a later time-point, administering a modulator of an apoptosis-related disease and a construct comprising a caspase-3/7 substrate attached to a luciferin molecule in a buffer comprising between about 30% PEG 300 or 400, about 5% DMSO, about 5%, Tween 80, and about 60% aqueous solution to the subject;
(l) creating a second image of the photon emission of the subject with a photodetector device; and
(m) comparing the photon emission of the first image and the second image.
